(19) United States
(12) Patent Application Publication
(10) Pub. No.: US 2013/0267842 A1
(43) Pub. Date: Oct. 10, 2013

(54) METHOD FOR OPERATING AN IMAGING DIAGNOSTIC DEVICE AND MEDICAL IMAGING SYSTEM

(71) Applicants: Michael Scheuering, Nurnberg (DE); Grzegorz Soza, Heroldsberg (DE)

(72) Inventors: Michael Scheuering, Nurnberg (DE); Grzegorz Soza, Heroldsberg (DE)

(21) Appl. No.: 13/854,272

(22) Filed: Apr. 1, 2013

(30) Foreign Application Priority Data
Apr. 5, 2012 (DE) 102012205711.0

(51) Int. Cl. A61B 6/00

(52) U.S. Cl. CPC A61B 6/481; A61B 6/5205
USPC 600/431

(57) ABSTRACT

A method for operating an imaging diagnostic device is proposed. Raw data is generated by the diagnostic device as a function of modality parameters while utilizing a contrast medium that is used in accordance with an injection protocol. Image data is generated from the raw data by image reconstruction as a function of reconstruction parameters. A quantitative measurement is performed in an image data evaluation by an analysis application. A subsidiary application automatically proposes a set of parameter values which is suited to the analysis application and relates to the injection protocol utilizing stored comparison data.

### CROSS REFERENCE TO RELATED APPLICATIONS

This application claims priority of German application No. 10 2012 205 711.0 filed Apr. 5, 2012, which is incorporated by reference herein in its entirety.

### FIELD OF INVENTION

The application relates to a method for operating a medical imaging diagnostic device, also referred to generally as a modality, such as a computed tomography system, and a medical imaging system for performing said method.

### BACKGROUND OF INVENTION

DE 10 2010 043 849 B3 discloses a computed tomography system for determining and depicting the perfusion of the heart muscle on the basis of statistical CCTA recordings (coronary CT angiography). In this context, provision is made for a segmentation unit for segmenting the coronary arteries and the left-hand heart muscle in a CCTA image of the heart, a first simulation unit for simulating the blood flow through the coronary arteries, and a second simulation unit by which the local perfusion can be determined on the basis of the blood flow that has been ascertained in different regions of the heart muscle. As part of the examination that is performed by the computed tomography system, a contrast medium is used in accordance with a patient-specific injection protocol.

Basic information relating to computed tomography systems and to spiral computed tomography can be found e.g. in the book entitled “Bildgebende Systeme für die medizinische Diagnostik” [Imaging Systems for Medical Diagnostics], 3rd edition, 1995, published by Heinz Morneburg (ISBN 3-89578-002-2; see section 5.5).

In the subsequently published German patent application 10 2011 078 278.8, a method is described for image generation and image evaluation in the medical field, wherein raw data is generated by a medical modality, specifically a computed tomography system, as a function of predetermined modality parameters. With the aid of image reconstruction, the raw data is used to generate image data as a function of predetermined reconstruction parameters, which image data is evaluated by an analysis application. A subsidiary application automatically proposes a set of parameter values, which is suited to the analysis application and/or predetermined patient information, for the modality parameters and/or reconstruction parameters.

In principle, images generated by imaging diagnostic devices such as computed tomography systems or magnetic resonance tomographs offer the possibility of quantitative image evaluation. Such an evaluation makes it possible to determine dimensions of adjacent regions identified within an examination object, for example.

### SUMMARY OF INVENTION

The object of the application is to further develop a method for operating an imaging diagnostic device relative to the cited prior art, such as with respect to the possibilities of quantitative image evaluation.

This object is achieved according to the application by a method and by a medical imaging system having the features in independent claims. Embodiments are the subject matter of the dependent claims.

The method according to the application has features as follows:
raw data is generated by a medical imaging diagnostic device (such as a computed tomography system) as a function of modality parameters, while utilizing a contrast medium that is used in accordance with an injection protocol,
image data is generated from the raw data by image reconstruction as a function of reconstruction parameters,
the image data is evaluated by an analysis application, wherein a quantitative measurement is performed,
utilizing stored comparison data, a subsidiary application automatically proposes a set of parameter values which is suited to the analysis application and relates to at least the injection protocol.

The application takes as its point of departure the idea that the results of quantitative measurements, which are performed in the context of imaging diagnostics and can play a key role in the diagnosis, are dependent on numerous factors of influence, and this can adversely affect the comparability of measurements performed at different time points. In order to obtain an objective assessment of the change in structures that were recorded at lengthy time intervals by an imaging diagnostic device, it is for the conditions under which the recordings were made to remain as constant as possible. Even when the conditions governing the acquisition of image data remain the same, restrictions often apply to the comparability of evaluation results. This may be due to evaluations being performed by various people, for example, who interpret structures recorded by the diagnostic device differently.

Even though it is impossible to eliminate or compensate for all of the influences that adversely affect the comparability of quantitative measurements, it is possible to minimize the variability of the evaluation of diagnostic image data by technical measures. In the case of examinations that are performed using a contrast medium, the parameters relating to the contrast medium injection also play a role in this context. According to the application, parameter values which relate to the injection protocol governing the use of the contrast medium are automatically proposed accordingly. In this case, the proposal for the parameters relating to contrast medium use is based firstly on the current examination to be performed by the imaging diagnostic device and subsequent evaluation, and secondly on stored comparison data. A subsidiary application is understood generally to be an algorithm by which existing data can be evaluated automatically and suitable parameter values that are derived can be determined and at least proposed.

The comparison data which is used to determine the parameters for the contrast medium injection can be retrieved from a medical information system, such as a radiology information system (RIS), and does not necessarily refer to the patient who is currently being examined by the imaging diagnostic device.

In an embodiment, provision is however made for using comparison data (if available) that was obtained during a previous examination of the same examination object, i.e. typically a patient. In this context, data must be used that was obtained in the context of a previous examination having the same indication as in the current examination, but not necessarily using the same diagnostic device or a diagnostic device of the same type.

The comparison data, which comes from at least one previous examination, typically comprises the type of the diagnostic device that was used for the previous examination, the scan protocol that was used, the injection protocol of the contrast medium, and the name of the person who performed the examination.

At least extracts of the comparison data are displayed to the operator of the diagnostic device. The parameter settings that are proposed for the current examination and relate to the contrast medium use are likewise displayed to the operator. In the simplest case, the injection protocol remains unchanged relative to the previous examination. If due to a changed factor the previous injection protocol would no longer result in comparable image data that could be quantitatively evaluated, new contrast medium injection parameters are derived automatically and stored in an updated injection protocol and displayed. Changed factors that must be taken into consideration when a parameter set is automatically proposed for the contrast medium injection include the weight, the height, the heart rate and the position of a patient, for example.

Likewise, the insertion point of an injection needle that is used to apply the contrast medium, if known from a previous examination, is taken into consideration when determining parameters for the current use of a contrast medium and displayed to the operator of the diagnostic device. If it is not possible to insert the injection needle at the same point as was used in the previous examination, e.g. due to changes resulting from chemotherapy, the person performing the examination has to determine a new insertion point and record this data. Taking this information into consideration, the diagnostic device performs a new calculation of the injection parameters on the basis of a parameterized anatomical patient model. According to the application, consideration is given to the sections along which the blood flows and the speed at which it flows, in order to determine the correct time delay between the introduction of the contrast medium and the start of the data recording by the diagnostic device. Once determined, the adapted parameters are clearly displayed to the operator as an injection protocol. When determining the set of parameter values, consideration is given generally to any geometric differences that may be present, e.g. the position of the insertion point or the section length that must be covered when utilizing the contrast medium at different time points.

If the operator of the diagnostic device selects parameters which are not proposed, provision is made for automatically checking the comparability of quantitative measurements that are based on image data obtained at various time points. If the various quantitative measurements are not comparable, or are only comparable to a limited extent, an embodiment automatically generates a warning message.

According to an embodiment, in addition to the parameters relating to the contrast medium injection, the automatically proposed set of parameter values also comprises modality parameters, i.e. parameters relating to the operation of the imaging diagnostic device. Such parameters are used to set a primary and/or secondary collimator, the scan mode, the scan duration or the scan speed, for example. The scan mode can be a spiral scan, for example, or a scan that is based on the step-and-shoot method.

Additionally or alternatively, the set of parameter values can comprise reconstruction parameters, i.e. parameters that are applied during the image reconstruction. The parameter values for image reconstruction can depend inter alia on contrast and sharpness requirements, it being taken into consideration that the sharpness of the image and the noise cannot normally be varied independently of each other in the context of image reconstruction and that these variables are weighted.

In all cases, irrespective of whether the set of parameter values comprises only parameters relating to the contrast medium injection, or additionally modality parameters, or additionally reconstruction parameters, or additionally both modality and reconstruction parameters, the automatic determination of the relevant parameters is effected by the subsidiary application on the basis of a user-selected analysis application which includes a quantitative measurement.

The analysis application is based firstly on the generation of raw data by the imaging diagnostic device, wherein modality parameters and the injection protocol must be specified. The analysis application is further based on the image data which is generated from the raw data using reconstruction parameters that have been set. When planning the examination and subsequent data evaluation, the user of the imaging diagnostic device first considers the analysis application, such as the quantitative measurement that is to be performed in the context of said application. The quantitative measurement can be purely a measurement of geometric features, a determination of the type or concentration of matter, e.g. tissues or deposits, or a combined determination of geometric and material properties.

According to a variant of the method, the operator can select the analysis application (postprocessing application) from a limited number of analysis applications. Following selection of the analysis application, which is typically realized in the form of software, the set of parameter values to be proposed is automatically determined by the subsidiary application with reference to the patient-specific comparison data.

Once the parameter values have been confirmed or modified by the operator, the actual examination by the imaging diagnostic device is started. It is that all of the steps involved in the whole process of data acquisition and processing through to the quantitative measurement have already been coordinated with each other before the examination starts, and that the parameters to be selected for the individual steps have been specified appropriately.

In an appropriate embodiment, an overview scan is initially performed by the imaging diagnostic device before the actual examination. Provision is made for automatically setting the region that is to be examined (field of view), this likewise being determined automatically on the basis of an image evaluation. In this way, it is possible to identify e.g. positional changes relative to a previous examination of the organ to be examined, before the actual examination using the contrast medium. The region to be scanned by the imaging diagnostic device is generally set specifically to the region of interest within the body.

### BRIEF DESCRIPTION OF THE DRAWINGS

An embodiment of the application is explained in greater detail below with reference to a drawing, in which:

FIG. 1 shows a schematic illustration of a medical imaging system comprising an imaging diagnostic device and connected data processing system,

FIG. 2 shows a flow diagram of a method that can be performed using the diagnostic device as per FIG. 1.

### DETAILED DESCRIPTION OF INVENTION

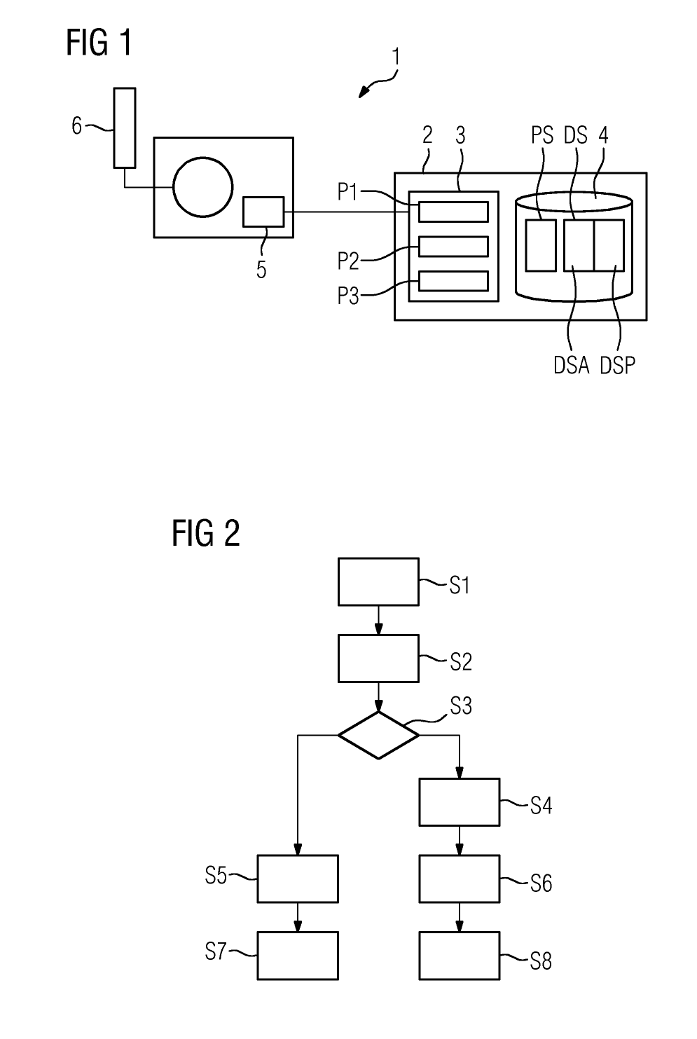

A medical diagnostic device 1, specifically a computed tomography system, which is merely outlined in FIG. 1 and whose principal function is described by the prior art cited in the introduction, has a data connection to a data processing system 2. The data processing system 2 can theoretically be realized as a single data processing device in the simplest case. However, the data processing system 2 is in fact designed as a data processing network which is connected to a radiology information system (RIS) or embedded in such a system.

The data processing system 2 comprises a computing unit 3 and a data store 4, wherein (as explained above) the schematic illustration according to FIG. 1 does not imply any hardware structures. The data store 4 has various storage areas, specifically a parameter store PS and an archive data store DS. The archive data store DS is in turn divided into a general data store DSA and a patient-specific data store DSP.

The computing unit 3 is designed to perform various functions, specifically the reconstruction of image data from raw data that is obtained by the imaging diagnostic device 1, and the analysis of said image data including a quantitative measurement. Various program modules can be executed by the computing unit 3, specifically a first program module P1 for the image reconstruction, an analysis application as a second program module P2 (allowing the quantitative measurement), and a subsidiary application as a third program module P3.

An internal computer 5 is integrated in the diagnostic device 1 and, in a manner that is comparable to the data processing system 2, allows both processing and storage of data. A contrast medium dispenser 6 is also provided, such that examinations can be performed by the diagnostic device 1 using a contrast medium.

A method that can be performed using the apparatus according to FIG. 1 is explained in detail below with reference to FIG. 2.

It is assumed that a patient has been taken to a medical facility and that the patient data is recorded first, before the patient undergoes x-ray examination by the diagnostic device 1 including dispensation of a contrast medium. In an emergency, it is also conceivable for the patient to be taken directly to the diagnostic device 1. In any case, the actual examination of the patient begins with the first method step designated S1.

It is already necessary in this step S1 to specify which analysis of image data will be performed. For this purpose, the operator of the diagnostic device 1 is offered a corresponding selection menu. Using the selection menu, the operator first specifies e.g. which region of the body is to be examined. Specifications may also be required in respect of the type of tissue that will be the subject of subsequent quantitative measurement.

In addition to the type of examination that is to be performed, further data (such as patient data) is relevant to the setting of parameters for the examination. For this, the diagnostic device 1 accesses the patient data management system in the medical facility, i.e. the data store 4. As part of this activity, a check establishes whether any data relating to the same indication as the present case, possibly an emergency, is actually available for the patient who is to be examined. If such data exists, it is usually retrieved from the patient-specific data store DSP.

In the case of emergencies, when a high-speed data connection to the data store 4 cannot be established, it is alternatively possible to access (if available) patient-specific data which is held in the store of the internal computer 5 of the diagnostic device 1.

Irrespective of the storage format and location of patient-specific data such as age, height, weight and the results of previous examinations, and the parameter settings that were applied during such examinations, the access to existing patient-specific data as comparison data is always the variant when providing support for the planning of the current examination.

If no patient-specific data exists, the general data store DSA is accessed instead. The general data store DSA comprises e.g. information that is available in a radiology information system, said information being based on simulations and/or on studies that have been conducted with large patient groups, and serving as comparison data. According to a development of the method, patient-specific data can also be linked to non-patient-specific data held in the general data store DSA, in order to support the selection of parameters for the examination that is to be performed.

The support for the selection of parameters is provided in the form of software in each case by the subsidiary application P3, which interacts with the analysis application P2 and, in the next method step S2, automatically generates a proposed specification of the parameters for the examination and for the subsequent image reconstruction.

The proposed set of parameter values comprises modality parameters PM, image reconstruction parameters PB (also referred to simply as reconstruction parameters), and injection parameters PI relating to the operation of the contrast medium dispenser 6. If the patient-specific data has not changed significantly in comparison with a previous examination and if the type of examination and evaluation including quantitative measurement are also approximately identical to the previous examination and evaluation, the subsidiary application P3 will propose that the parameter settings PB,PI,PM archived in the parameter store PS should also be used unchanged for the current examination and evaluation.

However, if the subsidiary application P3 ascertains that the comparability of the examinations (and associated quantitative measurements) that were performed or will be performed at various time points can be improved by a modified specification of the parameters PB,PI,PM, the analysis application proposes a correspondingly adapted specification of the parameters PB,PI,PM, likewise in the method step S2.

Irrespective of whether it is proposed that the parameters PB,PI,PM should be transferred unchanged in comparison with a previous examination, or a parameter modification is proposed, the operator of the diagnostic device 1 including contrast medium dispenser 6 must specify in the method step S3 whether the proposed parameters PB,PI,PM will be used. If not, the operator has the opportunity to change the parameters PB,PI,PM at this point in the method.

In the former case, i.e. if the proposed parameter setting is transferred, the method continues at the step S5 (left-hand branch in the flow diagram according to FIG. 2). In this method step S5, the actual imaging examination including dispensation of the contrast medium is performed by the diagnostic device 1.

In the case of at least partial modification of the proposed parameters PB,PI,PM, the method step S3 is followed by the step S4 (right-hand branch in the flow diagram according to FIG. 2). An automatic message is generated in this method step S4, informing the user of the parameter setting that deviates from the recommended setting of the parameters PB,PI,PM, such as in relation to the operation of the contrast medium dispenser 6. If the parameter setting specified by the user results in lack of comparability or limited comparability of the planned quantitative measurement with a previous measurement, or if the selected parameter setting produces other disadvantages, the output message is formulated as a warning. Once the user has acknowledged the message, the method continues at the step S6, which represents the actual imaging examination and corresponds, excepting the alternative parameter setting, to the step S5 of the method variant in which the proposed parameters PB,PI,PM are transferred.

Completion of the data recording in the step S5 or step S6 is followed by the evaluation of the reconstructed image data by the analysis application P2 in the step S7 or S8 respectively, wherein a quantitative measurement is also performed. The resulting data is archived in the patient-specific data store DSP, such that it is available for future examinations using the diagnostic device 1 or other modality.

1. A method for operating an imaging diagnostic device, comprising:
generating raw data by the diagnostic device as a function of modality parameters while utilizing a contrast medium in accordance with an injection protocol;
generating image data from the raw data by image reconstruction as a function of reconstruction parameters;
evaluating the image data for performing a quantitative measurement by an analysis application; and
automatically proposing a set of parameter values suited to the analysis application and related to the injection protocol by a subsidiary application utilizing stored comparison data.

2. The method as claimed in claim 1, wherein the stored comparison data was obtained in a previous examination of a same examination object.

3. The method as claimed in claim 1, wherein extracts of the stored comparison data are displayed before the set of parameter values are determined.

4. The method as claimed in claim 1, wherein a plurality of quantitative measurements are performed based on image data obtained at various time points, and wherein a comparability of the quantitative measurements is automatically checked if selected parameter values are differ from the proposed set of parameter values.

5. The method as claimed in claim 4, wherein a warning message is automatically generated if the quantitative measurements are comparable to a limited extent.

6. The method as claimed in claim 1, wherein the set of parameter values is determined according to geometric differences as a result of utilizing the contrast medium at various time points.

7. The method as claimed in claim 1, wherein the proposed set of parameter values comprises modality parameters.

8. The method as claimed in claim 1, wherein the proposed set of parameter values comprises reconstruction parameters.

9. The method as claimed in claim 1, further comprising selecting the analysis application from a plurality of analysis applications and automatically proposing the set of parameter values with reference to the stored comparison data.

10. A computer program product, comprising:
a program code to be executed on a data processing system for performing a method as claimed in claim 1.

11. A medical imaging system, comprising:
a diagnostic device; and
a data processing system connected to the diagnostic device that is adapted to perform a method as claimed in claim 1.
